(19) United States
(12) Patent Application Publication
(10) Pub. No.: US 2006/0068458 A1
(43) Pub. Date: Mar. 30, 2006

(54) Coupled enzymatic reaction system using a formate dehydrogenase derived from candida boidinii

(75) Inventors: Harald Groger, Hanau (DE); Werner Hummel, Titz (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA AG, Dusseldorf (DE)

(21) Appl. No.: 10/521,456

(22) PCT Filed: Jul. 7, 2003
(86) PCT No.: PCT/EP03/07246

§ 371 Date: Jan. 18, 2005

(30) Foreign Application Priority Data
Jul. 20, 2002 (DE) 102 33 046.8

(51) Int. Cl. C12Q 1/26

(52) U.S. Cl. 435/25

(57) ABSTRACT

The present process relates to a coupled enzymatic reaction system which is implemented in a two-phase system consisting of organic phase and aqueous phase. The system operates with cofactor-dependent enzymes, the cofactor being continually regenerated enzymatically by an FDH derived from C. boidinii.

The present invention relates to a coupled reaction system operating enzymatically which is distinguished in that it operates in a solvent mixture having two phases. In particular, the invention is directed towards a reaction system comprising a cofactor-dependent enzymatic transformation of an organic compound and an enzymatic cofactor regeneration using a formate dehydrogenase derived from Candida boidinii (or mutants based thereon) in the same system.

The isolation of optically active organic compounds, for example alcohols and amino acids, by biocatalytic means is gaining increasingly in importance. The coupled use of two dehydrogenases with cofactor regeneration has proved to be a method for the large-scale industrial synthesis of these compounds (DE 197 53 350).

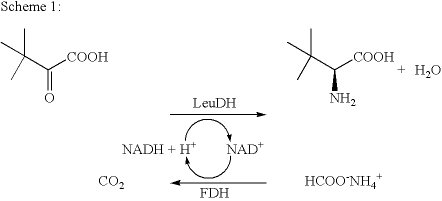

In situ regeneration of NADH with the NAD-dependent formate dehydrogenase derived from Candida boidinii in the course of the reductive amination of trimethyl pyruvate to L-pseudoleucine (Bommarius et al. Tetrahedron Asymmetry 1995, 6, 2851-2888).

Besides their catalytic property and efficiency, the biocatalysts that are employed efficiently in the aqueous medium additionally have the advantage, in contrast with a large number of synthetic metalliferous catalysts, that the use of metalliferous feed materials, in particular feed materials that contain heavy metal and are consequently toxic, can be dispensed with. The use of expensive and, in addition, hazardous reducing agents such as borane, for example, in the course of the asymmetric reduction can also be dispensed with.

However, difficulties arise in the course of the conversion of substrates that are sparingly soluble in water. Similar difficulties exist in connection with sparingly water-soluble products. This is the case, in particular, in the preparation of optically active alcohols in accordance with the above concept, since the ketones that are required as starting compounds have a clearly lower solubility than the α-keto acids employed in Scheme 1.

One conceivable solution in principle would be the implementation of the biocatalytic reduction using an alcohol dehydrogenase and a formate dehydrogenase in a polar organic solvent or in an aqueous solution thereof. In this case, both the enzymes and the substrate and optionally the product should be soluble. A general disadvantage of a direct presence of an organic solvent, however, is constituted by the considerable diminution of the enzyme activity which generally occurs under these conditions (see, for example, Anderson et al., Biotechnol. Bioeng. 1998, 57, 79-86). In particular, the formate dehydrogenase derived from Candida boidinii, being the only NADH regeneration enzyme employed hitherto on an industrial scale and available in commercial quantities, regrettably exhibits high sensitivity to organic solvents (EP 1 211 316). This is also shown in Comparative Example 1 with the use of DMSO, sulfolane, MTBE, acetone, isopropanol and ethanol etc. by way of organic solvent component in supplemental amounts of, in each case, only 10% by volume (see FIG. 1).

Various approaches are known with a view to solving this problem relating to a stabilisation of the formate dehydrogenase derived from Candida boidinii in the presence of organic solvents, for example the implementation of reactions through additional use of surfactants by way of surface-active substances. But the rate of reaction that is diminished by a factor of approximately 40 (!) and also the inhibition of the formate dehydrogenase which occurs (B. Orlich et al., Biotechnol. Bioeng. 1999, 65, 357-362.) prove to be disadvantageous in this case. The authors note in addition that, by reason of the low stability of the alcohol dehydrogenase, a reduction process under these conditions of a microemulsion is not economic. The same also holds, in principle, for the method described in EP 340 744, in which lyotropic mesophases were chosen as reaction site in the presence of an aqueous and/or organic phase.

A further basic possibility for the implementation of biocatalytic reactions consists in the application of immobilised enzymes in the organic solvent or the use of enzymes in a homogeneous solution consisting of water and a water-miscible organic solvent. However, the successes with these techniques, in which a direct contact of organic solvent and enzyme occurs, are limited to a few enzyme classes, in particular hydrolases. For instance, in DE 44 36 149 it is noted that the “direct presence of organic solvents (water-miscible or non-water-miscible) is tolerated only by a few enzymes that pertain to the class of the hydrolases.” Although a few further examples from other enzyme classes have become known in the meantime (inter alia, oxynitrilases and an FDH derived from yeast), the statement made in DE 44 36 149 continues to have validity for the majority of enzymes. For instance, an efficient immobilisation of the FDH derived from Candida boidinii is not known. In addition, the immobilisation itself is associated with additional costs due to the immobilisation step as well as the immobilisation materials.

Therefore processes have been developed industrially that avoid the presence of organic solvents by reason of the risk of the deactivation or denaturation of the enzymes. For instance, DE 44 36 149 describes a process in which the product is extracted from the reaction solution into an organic solvent through a product-permeable membrane, in particular a hydrophobic membrane. Compared with a standard process in a stirred-tank reactor, however, this process is technically clearly more elaborate; besides, the requisite organic membranes are also an additional cost factor. Furthermore, this method is only suitable for continuous processes. Moreover, it is a disadvantage that the achievable space-time yields with this procedure are comparatively low. For example, in the course of the reduction of acetophenone a space-time yield of only 88 g/(L*d) is obtained (S. Rissom et al., Tetrahedron: Asymmetry 1999, 10, 923-928). In this regard it is to be noted that acetophenone itself is a relatively well water-soluble ketone, and most analogue substituted acetophenone ketones and related ketones possess far lower solubilities, so that the space-time yield for typical hydrophobic ketones should be distinctly lower. Despite these considerable disadvantages, this process is regarded as the hitherto preferred method for the asymmetric biocatalytic reduction of sparingly soluble ketones using isolated enzymes (see also: A. Liese, K. Seelbach, C. Wandrey, Industrial Biotransformations, Wiley-VCH Verlag, Weinheim, 2000, pp. 103-106).

To sum up, it may consequently be noted that at the moment no process is known that helps to circumvent the disadvantages listed above and permits the enzymatic preparation of sparingly water-soluble substrates on an industrial scale using a formate dehydrogenase derived from Candida boidinii (or mutants based thereon) in the “direct” presence of organic solvents.

The object of the present invention was therefore to specify a possibility as to how, in particular, sparingly water-soluble organic compounds can be made available to a coupled cofactor-dependent enzymatic conversion to such a sufficient extent that an application of the conversion on an industrial scale can be undertaken under economically and ecologically advantageous conditions. In particular, one object was that such a process should be suitable for the reduction of sparingly water-soluble ketones and should permit the use of the formate dehydrogenase derived from Candida boidinii in the “direct” presence of organic solvents (i.e. without separation by a hydrophobic membrane).

This object is achieved in the manner defined in the claims. Claims 1 to 8 are directed towards a reaction system operating in accordance with the invention. Claim 9 protects a device. Claim 10 relates to a process operating in accordance with the invention, whereas claims 11 and 12 are directed towards preferred uses of the reaction system according to the invention.

By virtue of the fact that a coupled enzymatic reaction system is made available comprising an NADH-dependent enzymatic transformation of an organic compound with an alcohol dehydrogenase and an enzymatic regeneration of the NADH with the formate dehydrogenase derived from Candida boidinii or mutants thereof in a two-phase solvent system in which an aqueous phase is in contact with a liquid organic phase, the solution to the stated object is attained, in particular in a manner that is surprising, by no means foreseeable and, according to the invention, particularly advantageous. Contrary to the opinion that can be deduced from the state of the art, it is surprisingly possible, despite the presence of an organic solvent, to allow the coupled enzymatic reaction system to operate without loss of activity, due to the solvent, of one of the enzymes, in particular of the formate dehydrogenase derived from Candida boidinii, with space-time yields that are sufficient for the industrial scale. The FDH originates from the organism Candida boidinii itself, or further developed rec-mutants of the same organisms may also be employed (DE 197 53 350). Quite particularly advantageous is the use of the mutant that exhibits the C23S/C262A amino-acid substitutions. Particularly surprising in this connection is the fact that the formate dehydrogenase derived from C. boidinii, despite the high instability in relation to organic solvents that has been observed (see Comparative Example 1 in the Experimental Part), can be employed very efficiently under these conditions.

The organic solvent that is employed in the reaction system is intended to form—as explained above—two separate phases with the aqueous phase that is present. Within the bounds of this requirement, a person skilled in the art is, in principle, free in the choice of the organic solvent. However, it has proved to be advantageous if by way of organic phase a solvent is chosen that possesses a solubility in water that is as low as possible (logP value ≧3, preferably ≧3.1, more preferably ≧3.2 etc.). Since the organic solvent is also intended to take up the sparingly water-soluble educt at the same time, it is also important furthermore that said solvent possesses a solubility in respect of the organic compounds employed that is as high as possible.

Organic solvents of such a type, which can be preferentially employed in the reaction system, are aromatic or aliphatic hydrocarbons that are liquid under the given reaction conditions. In particular, n-pentane, n-hexane, n-heptane, n-octane, isooctane, cyclohexane, methylcyclohexane and also branched-chain isomers thereof are quite particularly preferred. Halogenated hydrocarbons can also be employed (CHCl₃, CH₂Cl₂, chlorobenzene etc.). By way of aromatic hydrocarbons, toluene, xylenes or benzene enter into consideration.

The quantitative ratio of organic solvent to aqueous portion can be chosen arbitrarily. The organic solvent is employed in a quantity relative to the total volume amounting to 5-80 vol. %, preferably 10-60 vol. %, particularly preferably around 50 vol. %.

Contrary to the approach that is proposed in the state of the art, namely of adding surfactants to the enzymatic reaction mixture in order to accelerate the enzymatic transformation, in which phase transitions in the course of the reaction are minimised, the present invention provides evidence that the use of a reaction system according to the invention proceeds particularly successfully when the system contains no surfactants.

The term ‘surfactants’ in this context is understood to mean all those substances which are capable of building up micellar structures or of lowering the surface tension at liquid-liquid phase boundaries.

As already indicated, the concentration with which the substrates are employed in the reaction system should be such that a conversion can be effected that is advantageous from economic viewpoints. The organic compound should therefore be present prior to the start of the reaction advantageously in a concentration of >25 mM, preferably >100 mM, particularly preferably >200 mM and quite particularly preferably >500 mM per L of total volume of the solvents (=sum of the volumes of organic solvent and aqueous portion). An upper limit for the concentration is constituted naturally by the guarantee of the viability of the reaction; in particular, stirrability of the reaction mixture should obtain in every case. However, working may preferably take place also above the saturation limit for the substrate or the product.

The concrete coupled enzymatic reaction system can, according to the invention, be employed in all enzymatic reactions coming into consideration by a person skilled in the art for this purpose in which keto groups are transformed into alcohol groups. Preferred, however, are oxidoreductase reactions, as stated. The process is suitable for the use of any types of alcohol dehydrogenase. The alcohol dehydrogenases that are employed in accordance with the invention preferably originate from the organisms Rhodococcus erythropolis (S-ADH) or Lactobacillus kefir (R-ADH) (ADH derived from R. erythropolis: J. Peters, T. Zelinski, M.-R. Kula, Purification and characterization of a novel carbonyl reductase silated from Rhodococcus erythropolis, J. Biotechnol. 1994, 33, 283-292) (ADH derived from Lactabacillus kefir: C. W. Bradshaw, W. Hummel, C.-H. Wong, Lactobacillus kefir Alcohol Dehydrogenase: A Useful Catalyst for Synthesis, J. Org. Chem. 1992, 57, 1532-1536.).

In a next development the present invention relates to a device for the transformation of organic compounds that comprises the reaction system according to the invention. Devices to be employed advantageously are, for example, the stirred tank or stirred-tank cascades, or membrane reactors that can be operated both in batch operation and continuously.

Within the scope of the invention the term ‘membrane reactor’ is understood to mean any reaction vessel in which the catalyst is enclosed in a reactor while low-molecular substances are supplied to the reactor or are able to leave it. In this connection the membrane may be integrated directly into the reaction chamber or may be installed outside in a separate filtration module wherein the reaction solution flows continuously or intermittently through the filtration module and the retentate is recycled into the reactor. Suitable embodiments are described, inter alia, in WO 98/22415 and in Wandrey et al. in Jahrbuch 1998, Verfahrenstechnik and Chemieingenieurwesen, VDI p 151 ff.; Wandrey et al. in Applied Homogeneous Catalysis with Organometallic Compounds, Vol. 2, VCH 1996, p 832 ff.; Kragl et al., Angew. Chem. 1996, 6, 684 f. The continuous mode of operation which is possible in this apparatus in addition to the batch and semicontinuous modes of operation can be implemented as desired in the cross-flow filtration mode (FIG. 4) or in the form of dead-end filtration (FIG. 3). Both process variants are described in principle in the state of the art (Engineering Processes for Bioseparations, Ed.: L. R. Weatherley, Heinemann, 1994, 135-165; Wandrey et al., Tetrahedron Asymmetry 1999, 10, 923-928).

A next development of the invention is concerned with a process for the enzymatic transformation of organic compounds by application of the reaction system according to the invention. The process is preferably one involving the preparation of an enantiomer-enriched organic compound, preferably a chiral alcohol. The design of the process can be worked out at the discretion of a person skilled in the art on the basis of the reaction system that has been described and the examples that are presented below. Under the given boundary conditions, the conditions that are otherwise known for the enzymatic conversion are set appropriately.

A next aspect of the invention is concerned also with the use of the reaction system according to the invention in a process for the enzymatic transformation of organic compounds or for the diagnosis or analysis of organic compounds, preferably of alcohols. In further preferred manner the reaction system according to the invention is, as stated, employed in a process for the preparation of enantiomer-enriched organic compounds, preferably of alcohols.

Surprisingly, the formate dehydrogenase (FDH) derived from Candida boidinii possesses very good stability in relation to two-phase solvent systems. This will be investigated on the basis of the experiments relating to the long-term stability of the FDH derived from C. boidinii in various solvent systems. In these experiments, according to Comparative Example 1 and also Example 2 according to the invention, a proportion of organic solvent of 10% and 20%, respectively, with respect to the total volume was chosen. In contrast with the water-soluble organic solvents (see Comparative Example 1), which lead to a rapid deactivation of the FDH derived from Candida boidinii, in the two-phase system, particularly when use is made of the aforementioned hydrocarbon components such as n-hexane, for example, outstanding stability properties of the formate dehydrogenase derived from C. boidinii (used in these Examples in the form of the double mutant) were still observed even after several days. Whereas, for example, in the presence of acetone or DMSO the enzyme activity declines within 24 hours by 35% or 66%, respectively, in the presence of 20 vol. % hexane 90% enzyme activity can still be registered even after 3 days. The results with n-hexane (Example 2) are reproduced in FIG. 1, represented graphically, and in Table 3. The Comparative Examples with other organic solvents are likewise recorded in FIG. 1 and Table 1.

According to the invention, other organic solvents that are not miscible with water and that consequently form two phases can also be employed in the process that has been described. For instance, it has been possible to achieve very high stabilities also with n-heptane by way of organic solvent component with a proportion of organic solvent of 20%. The stability after 27 hours in this case was an outstanding 99.8% (see Example 3 and Table 4). In totally surprising manner this activity is therefore still clearly above the activity of a pure aqueous solution, pointing to an unexpected stabilisation of the FDH derived from Candida boidinii through the use of a 2-phase system (see also Experimental Part, Example 3, Table 4 and FIG. 2).

Moreover, it is to be noted that the reaction system according to the invention permits the enzymatic conversions to be implemented successfully in the case where use is made of 2-phase systems with a higher organic proportion by volume. This is documented by the experiments that were carried out using n-heptane with a higher proportion of solvent (see Example 3, Table 4). In the case of a proportion by volume of the organic solvent n-heptane of 60%, a high activity over a long period could likewise be retained, as a residual activity of 82.8% after 27 hours makes clear. The results relating to the long-term stability with varying proportions by volume of organic solvent (according to Example 3, Table 4) are represented graphically in the illustration of FIG. 2.

The present invention can be elucidated on the basis of the example provided by the alcohol-dehydrogenase/NADH/FDH/formic-acid system. The asymmetric synthesis of alcohols was carried out by means of this reaction system, starting from the corresponding ketone.

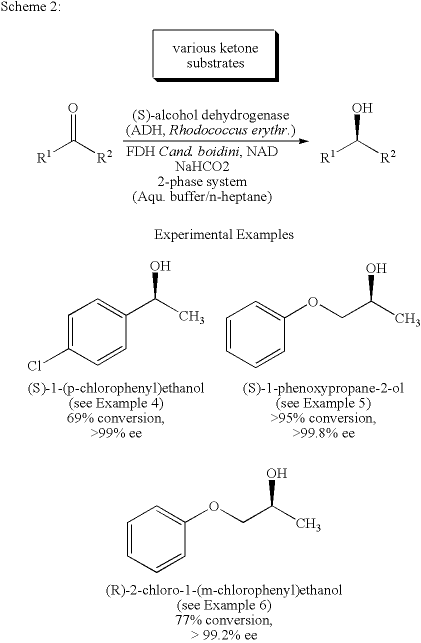

Processing of the reaction mixture was effected by extraction with MtBE and concentration of the organic phase by evaporation. The corresponding alcohol was obtained in this way in a very simple manner in terms of apparatus in a yield of 69% and with an enantioselectivity of 99% (Example 4).

But outstanding enantioselectivities are also obtained with the use of other ketones as starting materials. For instance, the reduction of phenoxyacetone under these reaction conditions results in an enantiopure product quantitatively with >99.8% ee (Example 5).

But the reaction system according to the invention is also suitable, moreover, for sterically demanding ketones. This will be documented in exemplary manner on the basis of the example provided by α,m-dichloroacetophenone. This ketone is substituted by a chlorine atom both on the methyl group and on the aromatic ring. The biocatalytic reduction in the 2-phase system here yields the desired product 2-chloro-1-(m-chlorophenyl)ethanol, again with outstanding enantioselectivity of >99.2% (Example 6). The conversion here is around 77%.

These high conversions and enantioselectivities are surprising, not least for the reason that, by virtue of the presence of organic solvents, often not only a diminution of the enzyme activity (accompanied by a low conversion) but also a change in enzyme properties with regard to stereospecificity (accompanied by a diminution of enantioselectivity) is to be observed.

In this context, however, the results of the experiments at elevated substrate concentrations turned out to be particularly surprising. These experiments were carried out with p-chloroacetophenone as model substrate. If in the above experiment at a substrate concentration of 10 mM (this substrate concentration corresponds to the concentration in the case of the experiments from the state of the art) a conversion of 69% is achieved (Example 4), then this conversion—contrary to the widespread view that at elevated substrate concentrations only diminished yields can be achieved, by reason of inhibitions etc.—was able to be increased with this type of reaction, starting now from a concentration of 20 mM, and higher conversions of 75% (at 40 mM) and 74% (at 100 mM) could be achieved (Examples 7, 8; Scheme 3; FIG. 5).

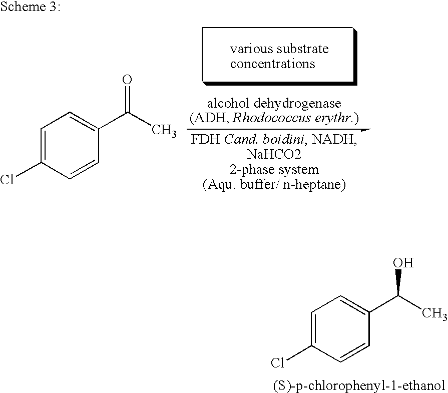

This process is consequently also suitable, in particular, for the enzymatic reduction of ketones at high substrate concentrations.

A principal advantage of this process consists in its simplicity. For instance, no elaborate process steps are included, and the process can be implemented both in batch reactors and continuously. Similarly, in contrast with earlier processes, no special membranes which separate the aqueous medium from the organic medium are required. The additions of surfactant which are required in some previous processes also become unnecessary with this process. A further principal advantage consists in the first-time possibility of organising the enzymatic preparation of optically active alcohols in technically meaningful substrate concentrations of >25 mM. These advantages could not be deduced in obvious manner from the state of the art.

The term ‘enantiomer-enriched’ designates the fact that one optical antipode is present in the mixture with its other one in a proportion amounting to >50%.

In the case where one stereocentre is present, the structures that are represented relate to both of the possible enantiomers and, in the case where more than one stereocentre is present in the molecule, to all possible diastereomers and, with respect to one diastereomer, to the possible two enantiomers of the compound in question which are encompassed thereby.

The organism C. boidinii is deposited in the American Type Culture Collection under number ATCC 32195 and is publicly accessible.

The expression ‘coupled enzymatic system’ is understood to mean, according to the invention, that an enzymatic transformation of an organic compound takes place subject to consumption of a cofactor and the cofactor is regenerated in situ by a second enzymatic system (here the FDH derived from C. boidinii or mutants thereof). As a result, this leads to a diminution of the use of expensive cofactors.

The documents of the state of the art that have been named in this publication are considered as being jointly encompassed by the disclosure.

### DESCRIPTIONS OF THE DRAWINGS

FIG. 3 shows a membrane reactor with dead-end filtration. The substrate 1 is transferred into the reactor chamber 3, which exhibits a membrane 5, via a pump 2. Located in the stirrer-driven reactor chamber, in addition to the solvent, are the catalyst 4, the product 6 and unconverted substrate 1. Principally low-molecular product 6 is filtered off via the membrane 5.

FIG. 4 shows a membrane reactor with cross-flow filtration. The substrate 7 here is transferred via the pump 8 into the stirred reactor chamber in which solvent, catalyst 9 and product 14 are also located. Via the pump 16 a flow of solvent is set which, via an optionally present heat-exchanger 12, leads into the cross-flow filtration cell 15. Here the low-molecular product 14 is separated via the membrane 13. High-molecular catalyst 9 is subsequently conducted back into the reactor 10 with the flow of solvent, optionally again via a heat-exchanger 12, optionally via the valve 11.

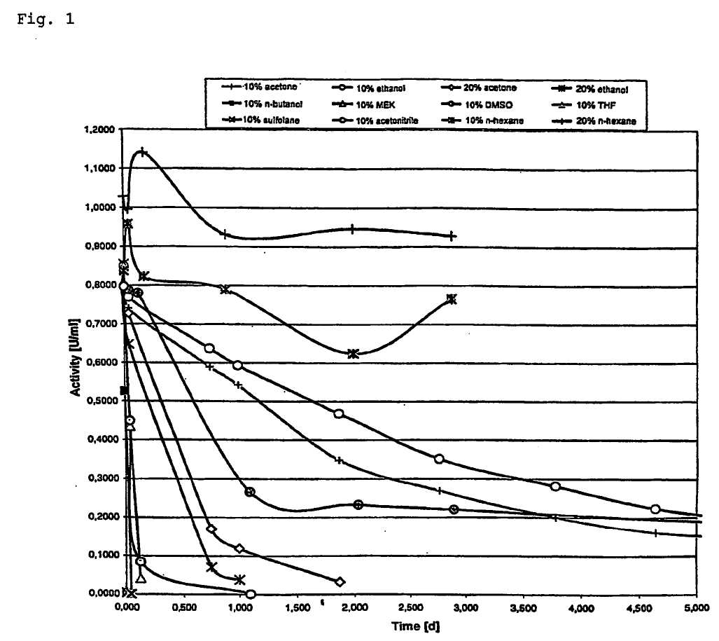

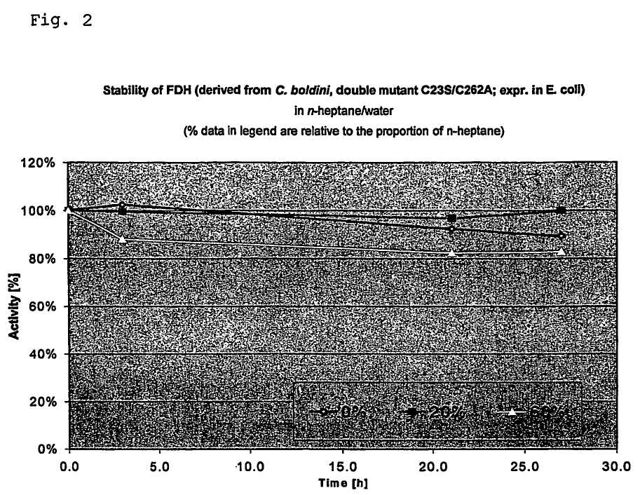

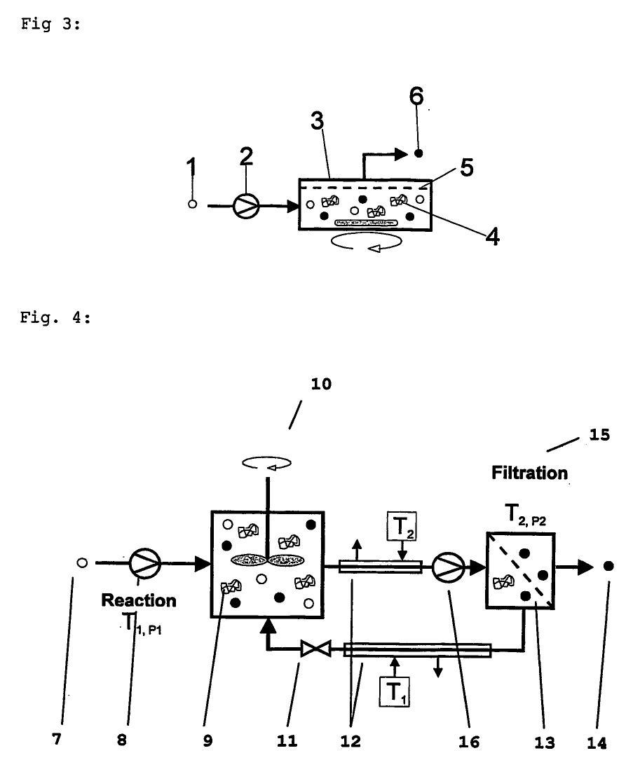

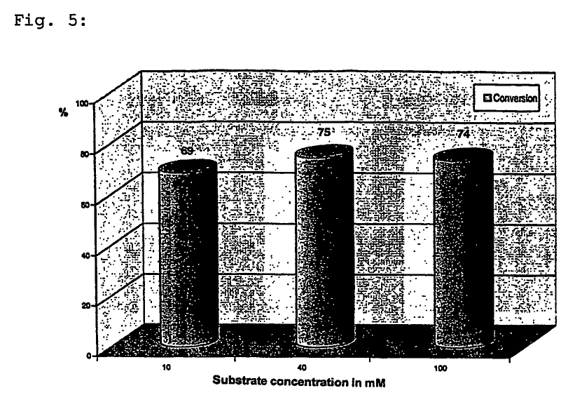

### EXPERIMENTAL PART

### Example 1

### Comparative Examples of the FDH Activities Using an FDH Derived from C. boidinii (Double Mutant: C23S/C262A)

2.72 g (0.8 mol/L) sodium formate and 1.14 g (0.1 mol/L) dipotassium hydrogenphosphate trihydrate are weighed out and dissolved in 40 mL fully demineralised H₂O. With ammonia solution (25%) and formic acid (100%), or corresponding diluted solutions, the pH value of the solution is set to 8.2. Then the solution is transferred into a 50 mL measuring flask and topped up with fully demineralised H₂O. Separately from this, 71.7 mg (4 mmol/L) NAD⁺ trihydrate are weighed out and dissolved in about 20 mL fully demineralised H₂O. With ammonia solution (25%) and formic acid (100%), or corresponding diluted solutions, the pH value of the solution is set to 8.2. Then the solution is transferred into a 0.25 mL measuring flask and topped up with fully demineralised H₂O. Subsequently, in each case, 500 μL of the substrate solution and also of the NADH solution are mixed in the 1 cm cell which is used for the measurement. After addition of 10 μL of the enzyme solution, whereby a 10% solution of an organic solvent (see Table) in water finds application by way of solvent, shaking is effected briefly, the cell is placed into the photometer, and the recording of data is started. The enzyme solution is firstly added directly prior to the start of measurement. The activities of the FDH derived from C. boidinii (double mutant: C23S/C262A) are determined after certain periods of time by the photometric detection of the reaction of NAD⁺ to form NADH. The photometric measurement was undertaken at a temperature of 30° C., at a wavelength of 340 nm and with a measuring-time of 15 min. The results are represented below in Table 1 and Table 2.

TABLE 1
Enzyme activity of the FDH derived from C. boidinii (double mutant:
C23S/C262A) in U/mL as a function of solvent and time

| | | | | | | Aceto- |
|---|---|---|---|---|---|---|
| | Butanol | MEK | DMSO | THF | Sulfolane | nitrile |
| Time | Activity | Activity | Activity | Activity | Activity | Activity |
| [d] | [U/ml] | [U/ml] | [U/ml] | [U/ml] | [U/ml] | [U/ml] |
| 0.000 | 0.5262 | 0.0058 | 0.7965 | 0.8492 | 0.0028 | 0.7961 |
| 0.042 | 0.0006 | 0.0011 | 0.7880 | 0.4357 | 0.0003 | 0.4494 |
| 0.125 | | | 0.7794 | 0.0414 | | 0.0840 |
| 1.097 | | | 0.2669 | | | 0.0008 |
| 2.035 | | | 0.2331 | | | |
| 2.896 | | | 0.2201 | | | |
| 5.927 | | | 0.1763 | | | |
| 7.885 | | | 0.1404 | | | |
| 9.948 | | | 0.1205 | | | |
| 13.073 | | | 0.0915 | | | |
| 14.892 | | | 0.0717 | | | |
| 16.875 | | | 0.0540 | | | |
| 19.938 | | | 0.0355 | | | |

Table 2
Enzyme activity of the FDH derived from C. boidinii (double mutant:
C23S/C262A) in U/mL as a function of solvent and time

| Time | Acetone | Ethanol |
|---|---|---|
| [d] | Activity [U/ml] | Activity [U/ml] |
| 0.000 | 0.8355 | 0.8491 |
| 0.042 | 0.7402 | 0.7689 |
| 0.750 | 0.5893 | 0.6367 |
| 1.000 | 0.5426 | 0.5933 |
| 1.875 | 0.3484 | 0.4687 |
| 2.760 | 0.2691 | 0.3510 |
| 3.781 | 0.2004 | 0.2814 |
| 4.646 | 0.1614 | 0.2240 |
| 5.875 | 0.1325 | 0.1736 |
| 6.778 | 0.0987 | 0.1486 |
| 7.792 | 0.0794 | 0.1277 |
| 8.729 | 0.0610 | 0.0998 |
| 11.750 | 0.0333 | 0.0536 |
| 13.726 | | 0.0421 |

### Example 2

### Measurement of the FDH Activities of the FDH Derived from C. boidinii (Double Mutant: C23S/C262A)

Determination of the activity was undertaken in accordance with the instructions in Example 1, with hexane being used as organic solvent component. The results are represented below in Table 3.

Table 3
Enzyme activity of the FDH derived from C. boidinii (double mutant:
C23S/C262A) in U/mL as a function of hexane and time

| Time | Hexane (10%) | Hexane (20%) |
|---|---|---|
| [d] | Activity [U/ml] | Activity [U/ml] |
| 0.000 | 0.8334 | 1.0280 |
| 0.042 | 0.9572 | 0.9952 |
| 0.177 | 0.8223 | 1.1408 |
| 0.899 | 0.7892 | 0.9311 |
| 2.000 | 0.6242 | 0.9467 |
| 2.878 | 0.7654 | 0.9280 |

### Example 3

### Measurement of the FDH Activities of the FDH Derived from C. boidinii (Double Mutant: C23S/C262A)

Determination of the activity was undertaken in accordance with the instructions in Example 1, with n-heptane being used as organic solvent component. The results are presented below in Table 4, the evaluation being given here in percentage terms and the results at the individual substrate concentrations relating in each case to the initial activity which is denoted by 100%.

Table 4
Enzyme activity of the FDH derived from C. boidinii (double mutant:
C23S/C262A) in U/mL as a function of hexane and time

| | n-Heptane | n-Heptane | n-Heptane |
|---|---|---|---|
| Time | (0%) | (20%) | (60%) |
| [h] | Activity [%] | Activity [%] | Activity [%] |
| 0 | 100.0 | 100.0 | 100.0 |
| 3 | 102.3 | 99.5 | 88.1 |
| 21 | 92.2 | 96.7 | 82.3 |
| 27 | 89.3 | 99.8 | 82.8 |

### Example 4

### Conversion with p-chloroacetophenone

To a solution consisting of p-chloroacetophenone (78.4 mg; 10 mM), sodium formate (50 mM) and NADH (2 mM) in 10 mL n-heptane and 40 mL of a phosphate buffer, 10.1 U of alcohol dehydrogenase (derived from Rhodococcus erythropolis) and 10 U of a formate dehydrogenase (FDH derived from C. boidinii, expression in E. coli, double mutant C23S/C262A) are added. The reaction mixture that has arisen is left to stir for 21 hours at 30° C. Subsequently processing proceeds via extraction with 3×25 mL MTBE, and the collected organic phases are dried with sodium sulfate. The crude product resulting after removal of the solvent in a vacuum is examined with regard to conversion (by ¹H-NMR spectroscopic examination) and enantioselectivity (by chiral GC).
Conversion: 69%
Enantioselectivity: >99% ee

### Example 5

### Conversion with Phenoxyacetone

To a solution consisting of phenoxyacetone (76.0 mg; 10 mM), sodium formate (50 mM) and NADH (2 mM) in 10 mL n-heptane and 40 mL of a phosphate buffer, 10.1 U of alcohol dehydrogenase (derived from Rhodococcus erythropolis) and 10 U of a formate dehydrogenase (FDH derived from C. boidinii, expression in E. coli, double mutant C23S/C262A) are added. The reaction mixture that has arisen is left to stir for 21 hours at 30° C. Subsequently processing proceeds via extraction with 3×25 mL MTBE, and the collected organic phases are dried with sodium sulfate. The crude product resulting after removal of the solvent in a vacuum is examined with regard to conversion (by ¹H-NMR spectroscopic examination) and enantioselectivity (by chiral GC).
Conversion: >95%
Enantioselectivity: >99.8% ee

### Example 6

### Conversion with 2,3′-dichloroacetophenone

To a solution consisting of 2,3′-dichloroacetophenone (102.7 mg; 10 mM), sodium formate (50 mM) and NADH (2 mM) in 10 mL n-heptane and 40 mL of a phosphate buffer, 10.1 U of alcohol dehydrogenase (derived from Rhodococcus erythropolis) and 10 U of a formate dehydrogenase (FDH derived from C. boidinii, expression in E. coli, double mutant C23S/C262A) are added. The reaction mixture that has arisen is left to stir for 21 hours at 30° C. Subsequently processing proceeds via extraction with 3×25 mL MTBE, and the collected organic phases are dried with sodium sulfate. The crude product resulting after removal of the solvent in a vacuum is examined with regard to conversion (by ¹H-NMR spectroscopic examination) and enantioselectivity (by chiral GC).
Conversion: 77%
Enantioselectivity: >99.2% ee

### Example 7

### Conversion with p-chloroacetophenone at 40 mM

To a solution consisting of p-chloroacetophenone (78.4 mg; 10 mM), sodium formate (50 mM) and NADH (2 mM) in 2.5 mL n-heptane and 10 mL of a phosphate buffer, 10.1 U of alcohol dehydrogenase (derived from Rhodococcus erythropolis) and 10 U of a formate dehydrogenase (FDH derived from C. boidinii, expression in E. coli, double mutant C23S/C262A) are added. The reaction mixture that has arisen is left to stir for 21 hours at 30° C. Subsequently processing proceeds via extraction with 3×25 mL MTBE, and the collected organic phases are dried with sodium sulfate. The crude product resulting after removal of the solvent in a vacuum is examined with regard to conversion (by ¹H-NMR spectroscopic examination) and enantioselectivity (by chiral GC).
Conversion: 75%

### Example 8

### Conversion with p-chloroacetophenone at 100 mM

To a solution consisting of p-chloroacetophenone (78.4 mg; 10 mM), sodium formate (50 mM) and NADH (2 mM) in 1 mL n-heptane and 4 mL of a phosphate buffer, 10.1 U of alcohol dehydrogenase (derived from Rhodococcus erythropolis) and 10 U of a formate dehydrogenase (FDH derived from C. boidinii, expression in E. coli, double mutant C23S/C262A) are added. The reaction mixture that has arisen is left to stir for 21 hours at 30° C. Subsequently processing proceeds via extraction with 3×25 mL MTBE, and the collected organic phases are dried with sodium sulfate.

The crude product resulting after removal of the solvent in a vacuum is examined with regard to conversion (by ¹H-NMR spectroscopic examination) and enantioselectivity (by chiral GC).
Conversion: 74%

1. A coupled enzymatic reaction system comprising an NADH-dependent enzymatic transformation of an organic compound with an alcohol dehydrogenase and an enzymatic regeneration of the NADH with the formate dehydrogenase derived from Candida boidinii or mutants thereof in a two-phase solvent system, wherein an aqueous phase is in contact with a liquid organic phase.

2. Reaction system according to claim 1,
characterised in that
the organic solvent employed possesses a solubility in water that is as low as possible and a solubility in respect of the organic compounds employed that is as high as possible.

3. Reaction system according to claim 1 and/or 2,
characterised in that
aromatic or aliphatic hydrocarbons that are liquid under the reaction conditions, in particular those having a logP value of >3, are employed as organic solvent.

4. Reaction system according to claim 1, 2 and/or 3,
characterised in that
the organic solvent is present in a quantity amounting to 10-60 vol. % in relation to the total volume.

5. Reaction system according to one or more of the preceding claims,
characterised in that
the organic compound is present prior to the start of the reaction in a concentration of >25 mM per L solvent mixture, in particular >100 mM per L solvent mixture.

6. Reaction system according to one or more of the preceding claims,
characterised in that
the system contains no surfactants.

7. Reaction system according to one or more of the preceding claims,
characterised in that
an alcohol dehydrogenase derived from Lactobacillus kefir is employed as enzyme for the transformation of the organic compound.

8. Reaction system according to one or more of claims 1 to 6,
characterised in that
an alcohol dehydrogenase derived from Rhodococcus erythropolis is employed as enzyme for the transformation of the organic compound.

9. A device for the transformation of organic compounds, comprising a reaction system according to ° claim 1.

10. A process for the enzymatic transformation of organic compounds by application of the reaction system according to claim 1.

11. Use of the reaction system according to claim 1 for the enzymatic transformation of organic compounds or for the diagnosis or analysis of, preferably, alcohols.

12. Use according to claim 11 in a process for preparing enantiomer-enriched organic compounds, preferably alcohols.
